(19) United States
(12) Patent Application Publication
(10) Pub. No.: US 2009/0149527 A1
(43) Pub. Date: Jun. 11, 2009

(54) Apparatus and Method for Application of a Pharmaceutical to a Surface of an External Ear Canal for Treatment of Keratosis Obutrans

(75) Inventor: Edward J. Timm, St. Louis, MO (US)

Correspondence Address: THOMPSON COBURN LLP, ONE US BANK PLAZA, SUITE 3500, ST LOUIS, MO, 63101, US

(73) Assignee: MOBIUS THERAPEUTICS, LLC, St. Louis, MO (US)

(21) Appl. No.: 12/371,716

(22) Filed: Feb. 16, 2009

Related U.S. Application Data
(63) Continuation-in-part of application No. 11/556,578, filed on Nov. 3, 2006, now Pat. No. 7,494,487.

(51) Int. Cl. A61K 31/407; A61M 5/32; A61B 19/02

(52) U.S. Cl. 514/410; 604/272; 206/572

(57) ABSTRACT

A packaged kit for treating keratosis obturans includes a plurality of ear needles having different shaped absorbent applicators on distal ends of the needles, a vial of a single dose of an otologic formulation of mitomycin-C, and a diluent carrier syringe containing sterilized water. The component parts of the kit are used together to reconstitute the contents of the vial with the water in the diluent carrier syringe. A selected one of the plurality of ear needles is then communicated with the syringe. The syringe and needle are then used to inject the reconstituted drug into the absorbent pad at the end of the needle, and the absorbent pad containing the drug is used to apply the drug to the surface of the external ear canal.

### REFERENCE TO RELATED APPLICATION

This patent application is a continuation-in-part of patent application Ser. No. 11/556,578, which was filed on Nov. 3, 2006, and is currently pending.

### BACKGROUND OF THE INVENTION

1. Field of the Invention

The present invention pertains to an apparatus and method for applying a pharmaceutical to interior tissue of an ear canal. In particular, the present invention pertains to a packaged kit that includes an ear needle having a flexible absorbent applicator on a distal end of the needle, a vial of a single dose of an otologic formulation of mitomycin-C, and a diluent carrier syringe containing sterilized water. The component parts of the apparatus are used together to reconstitute the contents of the vial with the water in the diluent carrier syringe. The ear needle is then attached to and communicated with the syringe. The syringe and needle are then used to inject the reconstituted drug into the absorbent applicator at the end of the needle, and use the absorbent applicator containing the drug to apply the drug to the surface of an external ear canal for the treatment of keratosis obturans at the surface.

2. Description of the Related Art

Mitomycin-C is an anti-metabolic agent that acts by interrupting DNA synthesis. It has been used as a chemotherapy agent, for example, in stomach and pancreatic cancers, for many years. Its anti-metabolic properties have prompted ophthalmologists to consider its use as a means of improving patency in trabeculectomy surgery. This procedure is well suited for the use of mitomycin, and the use of mitomycin in the procedure has ultimately become a standard of physicians.

The successful fistulae formation in glaucoma surgery with the accompanying use of mitomycin-C has resulted in experimentation in a variety of different surgical procedures where the desired end point, a functional, patent fistulae, is the same. Notable among these procedures is the myringotomy procedure, or the surgical creation of a pathway through the tympanic membrane. It has been contemplated that mitomycin-C could similarly sustain patency of a myringotomy such that the functional objective—middle ear ventilation and the resolution of chronic acute infection—is resolved without the implementation of pressure equalization tubes. This is described in the parent patent application Ser. No. 11/556,578, which is incorporated herein by reference.

A relatively rare otologic occurrence is keratosis obturans. Keratosis obturans occurs in one in one thousand new otologic patients and is characterized by a proliferative growth and accumulation of endothelial cells within the external ear canal, i.e. the portion of the ear canal that is external to the tympanic membrane. Over time, this accumulation of cells forms a plug that obstructs the external ear canal in its entirety, thereby suppressing the hearing sense of the affected patient. The only known treatment for keratosis obturans is the routine removal of the accumulated growth of endothelial cells by way of in-office, minor surgery. In this procedure, the physician grasps the plug of endothelial tissue and removes it from the ear canal. However, this treatment is only temporary, as the mechanism that creates this prolific growth of cells is unaffected by the removal of the plug of endothelial tissue. The endothelial cells will continue to grow and accumulate in the external ear canal, resulting in a reformation of the plug suppressing the hearing sense of the affected patient.

### SUMMARY OF THE INVENTION

It has been contemplated by the inventor that the concomitant use of mitomycin-C at the time of the removal of the prolific endothelial growth will limit its regrowth in the external ear canal and effectively treat the underlying psychological mechanism of keratosis obturans.

The mitomycin-C could be applied to the surface of the external ear canal by a sponge or some other absorbent, conformal delivery device, allowing for the uniform application of the anti-metabolic agent throughout the entirety of the affected pathology. The mitomycin-C could be reconstituted from lyophilized to liquid form at the time of the procedure, thereby minimizing issues of shelf life and substance fragility. It could be delivered to the external ear canal in a completely closed system, thereby minimizing inadvertent contact with a potentially hazardous material such as mitomycin-C. Finally, it could be prepared in a kit inclusive of proper disposal containment, thereby maximizing procedural convenience and further minimizing inadvertent, hazardous contact.

Thus, the present invention provides an apparatus and method for the application of a single dose of an otologic formulation of mitomycin-C to the surface of the external ear canal of a patient, where the mitomycin-C is suspended on the surface. Additionally, the mitomycin-C could be suspended on the surface by a viscous agent and/or an adhesive agent.

Still further, the apparatus and method of the invention employs a packaged kit that includes a vial of the mitomycin-C, a syringe that functions as a diluent carrier, and at least one ear needle having an absorbent, flexible applicator at the needle distal end. The diluent carrier syringe is used to extract the mitomycin-C from the vial into the syringe and mix the mitomycin-C with water in the syringe, forming a single dose of an otologic formulation of the mitomycin-C. The ear needle is then attached to the syringe and is used with the syringe to apply the mitomycin-C to the surface of the external ear canal following the removal of the plug of endothelial cells.

The apparatus of the invention and its method of use discussed above enable a safe and simplified treatment of keratosis obturans.

### DESCRIPTION OF THE DRAWING FIGURES

Further features of the invention are set forth in the following detailed description of the preferred embodiment of the invention and in the application drawing figures.

FIG. 1 is a perspective view of the component parts of the apparatus of the invention and of the packaging that combines the component parts as a kit in the packaging.

FIG. 2 is a perspective view of one of the ear needles of the apparatus.

FIGS. 3a and 3b are views of a cone-shaped absorbent applicator pad fixed on the distal end of one of the ear needles.

FIGS. 4a-d are views of a planar, kidney-shaped absorbent applicator pad fixed on the distal end of one of the ear needles.

FIG. 5 is a view of a cylinder-shaped absorbent applicator pad fixed on the distal end of one of the ear needles.

FIG. 6 is a side-sectioned view of one of the ear needles with the absorbent applicator pad removed from the distal end.

FIG. 7 is a sectioned representation of an inner ear canal.

FIG. 8 is a representation of a portion of the inner ear canal shown in the rectangle of FIG. 7.

FIG. 9 is a view similar to FIG. 8, but showing a pharmaceutical being applied to the surface of the external ear canal by the applicator shown in FIG. 5.

FIG. 10 is a view similar to FIG. 9, but showing the pharmaceutical being applied by another of the ear needles of the apparatus.

FIG. 11 is a view similar to FIG. 9, but showing the pharmaceutical being applied by another of the ear needles of the apparatus to the surface of the external ear canal.

FIG. 12 is a view similar to FIG. 9, but showing a laser beam ablating the tympanic membrane at a location treated by the pharmaceutical of the apparatus.

### DETAILED DESCRIPTION OF THE PREFERRED EMBODIMENT OF THE INVENTION

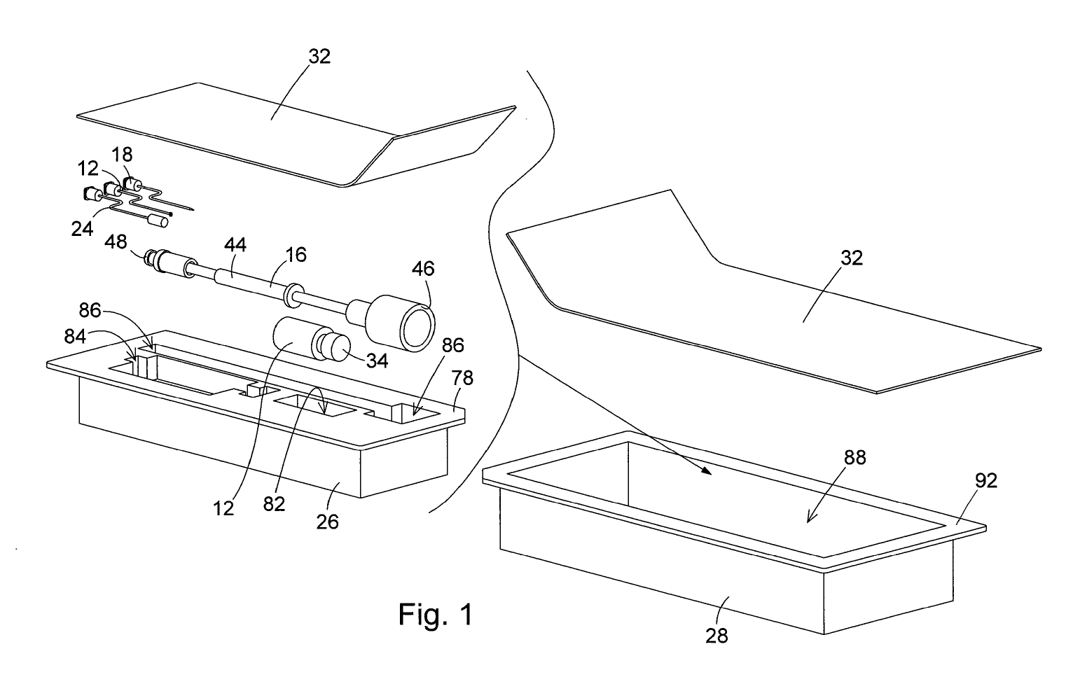

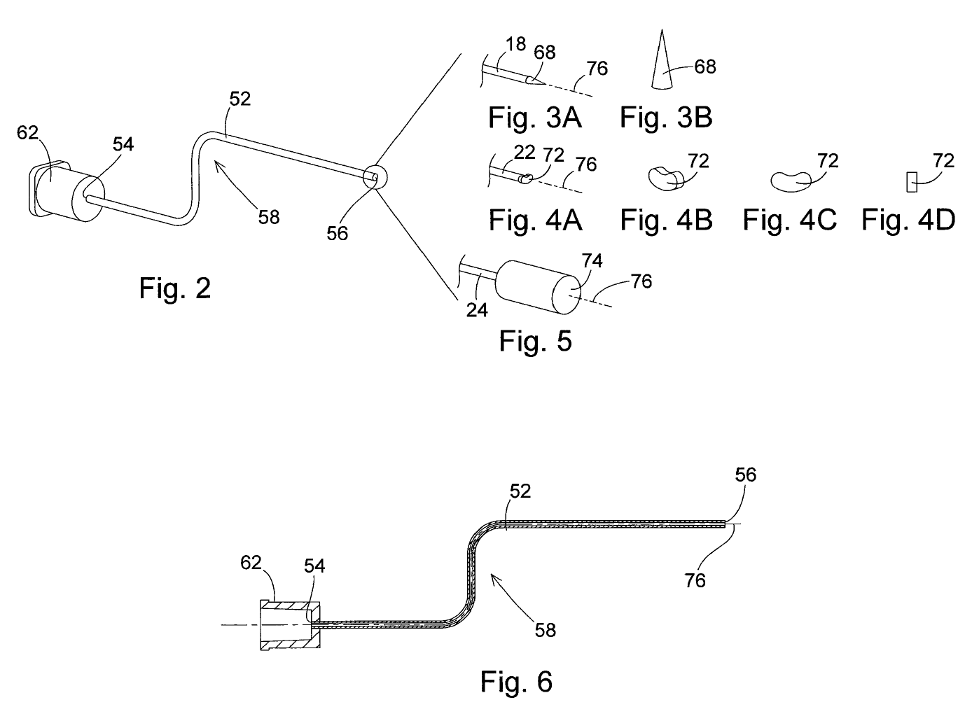

The component parts of the apparatus of the invention are shown in FIG. 1. These include the parts of the invention that enable the safe application of a pharmaceutical such as mitomycin-C to the surface of the external ear canal, and the assembling of the component parts after their use for safe disposal. Several of the component parts are known in the prior art in one form or another. Therefore, these component parts will be referred to by their commonly understood names, but will not be described in detail. The materials used to construct the component parts of the invention are those that are most typically used for constructing similar parts.

The component parts include a vial 12 of the pharmaceutical, a diluent carrier 16 that also functions as a syringe, a plurality of ear needles 18, 22, 24, a resilient packaging block 26, a semi-rigid packaging box 28, and a sheet of packaging material 32. As stated earlier, each of these component parts is constructed of materials typically used in manufacturing similar parts.

The pharmaceutical vial 12 has a construction that is known in the art. In the preferred embodiment, the pharmaceutical contained by the vial 12 is a single dose of an otologic formulation of mitomycin-C. The mitomycin-C has a specific color. Like conventional pharmaceutical vials, the vial 12 has a top 34 that can be pierced by a syringe needle which seals closed after the needle is removed from the top 34.

The diluent carrier 16 in the preferred embodiment of the invention is comprised of a syringe 44 containing a sterile liquid such as water, and a one-way valve 46, for example a Qosina-brand valve. However, other types of diluent carriers that perform the same function as the diluent carrier 16 to be described could be used in the apparatus kit. In the preferred embodiment, the diluent carrier 16 contains sterilized water. The amount of water provided in the carrier 16 is determined to mix with the pharmaceutical contained in the vial 12 to reconstitute the pharmaceutical in the carrier 16 to a desired therapeutic concentration. The carrier 16 has a proximal end 46 that is adapted to receive the vial top 34. A needle (not shown) is positioned in the carrier proximal end 46 to pierce the vial top 34 and communicate the pharmaceutical contained by the vial 12 with the water contained in the carrier 16. The carrier proximal end 46 also functions as the plunger of the carrier syringe 44. Moving the plunger proximal end 46 away from the syringe 44 creates a vacuum inside the syringe 44 that draws the pharmaceutical from the vial 12, through the one-way valve into the syringe 44. The one-way valve prevents the reconstituted pharmaceutical inside the syringe from returning to the vial when the plunger proximal end 46 is pushed back toward the syringe 44. The carrier distal end 48 is adapted to communicate with a selected one of the ear needles 18, 22, 24. As stated earlier, diluent carriers of this type are known in the art.

Each of the ear needles 18, 22, 24 have the same basic construction, the exception being the shape of the absorbent pad fixed to the needle. Each needle 18, 22, 24 is formed from a length of 20 gauge hypodermic tubing 52 with opposite proximal 54 and distal 56 ends. An intermediate portion 58 of the tubing is formed with pairs of right angles to adapt the tubing 52 for insertion into the ear canal. A standard syringe male luer 62 is secured to each needle proximal end 54. The male luer 62 is adapted to attach to the diluent carrier syringe 44 at the syringe distal end 46 to communicate the needle tubing 52 with the syringe body 44.

The three ear needles 18, 22, 24 differ from each other in that they have different shaped applicators 68, 72, 74 at the distal ends of the needles. In the preferred embodiment, each of the applicators 68, 72, 74 is an absorbent, flexible pad fixed to the distal ends of each of the needles 18, 22, 24. In the preferred embodiment, the pads 68, 72, 74 are constructed of an absorbent material that is known in the art and is used for the transient application of a pharmaceutical such as mitomycin-C. Each of the absorbent pads 68, 72, 74 is preferably constructed of polyvinyl acetate (PVA) sponge material. This material rapidly absorbs liquid such as the mitomycin-C. The dimensions of each pad 68, 72, 74 hold a specific volume of the mitomycin-C that enables a therapeutic, single dose of the otologic formulation of mitomycin-C to be applied to the tissue of an ear. Other types of applicators could also be used on the needle distal ends.

One of the absorbent pads 68 has a cone shape that projects from the needle distal end 56 to a tip of the cone. The cone shape of the pad 68 has a center axis 76 that is coaxial with a center axis 76 of the needle distal end 56. In the preferred embodiment, the cone-shaped pad 68 has a base diameter dimension of 0.04″, and an axial length dimension of 0.12″.

Another of the absorbent pads 72 has a planar configuration that extends transverse to the center axis 76 of the needle distal end 56. This pad 72 is formed in a general kidney shape. The pad 72 has a length dimension of 0.08″, and a width dimension of 0.04″. The pad 72 has a thickness of 0.02″.

A third pad 74 has a cylindrical configuration. The center axis of the pad cylinder 74 is coaxial with the center axis 76 of the needle distal end 56. In this embodiment of the pad 74, the pad has a diameter dimension of 0.2″ and an axial length dimension of 0.4″. This pad is intended to be used in the circumferential application of mitomycin-C to the walls of the external ear canal.

Each of the above-described component parts of the apparatus is contained in the packaging of the apparatus that includes the box 28, the resilient block 26, and the sheet of packaging material 32. Each of these packaging component parts is constructed of materials used in the safe storage, transport, and disposal of pharmaceuticals and instruments used with pharmaceuticals such as mitomycin-C.

The block 28 is constructed of a resilient material such as foam rubber or polystyrene. The block 28 has a top surface 78 that is formed with a plurality of cavities or compartments 82, 84, 86. Each of the compartments 82, 84, 86 is dimensioned to receive and securely hold the vial 12, the diluent carrier 16, and the plurality of ear needles 18, 22, 24. The compartments 82, 84, 86 securely hold the component parts of the apparatus and provide cushioning of the component parts to protect the parts during their storage and transportation.

The box 28 is dimensioned with an interior 88 that receives the resilient block 26 and securely holds the block 26 in the box interior. The box has top edges 92 that border the top opening to the box interior 88. The box 28 is dimensioned so that the top edges 92 will be positioned in the same plane as the top surface 78 of the resilient block 26 when the block is positioned in the box interior 88.

The sheet of packaging material 32 can be any type of material currently used to provide a sealed enclosure of the box 28. The packaging material 32 can be shrink-wrap applied around the box 28, or can be a resealable sheet of packaging material that can be peeled back from the box top edges 92 and then resealed to the top edges after the component parts of the apparatus have been removed from the packaging and used.

In use of the apparatus of the invention according to the method of the invention, the packaging is first opened by removing the sheet material 32 from the top edges 92 of the box. This exposes the block compartments 82, 84, 86 in the block top surfaces 78. The vial 12, the diluent carrier 16, and the ear needles 18, 22, 24 may be removed from their respective compartments in the resilient block 26.

The vial 12 of pharmaceutical, preferably mitomycin-C having a specific color, is then connected to the diluent carrier proximal end 46. This communicates the pharmaceutical in the vial 12 with the interior of the carrier 16. The pharmaceutical is drawn into the syringe 44 of the carrier 16 as explained earlier, and mixes with the water in the carrier 16, reconstituting the mitomycin-C. In addition, a viscous agent and/or an adhesive agent may be mixed with the mitomycin-C in the carrier 16.

A selected one of the ear needles 18, 22, 24 is next connected to the carrier syringe 44 distal end 48. The choice of the ear needle 18, 22, 24 is made by the physician determining which configuration of absorbent pad 68, 72, 74 is desirable for applying the mitomycin-C to the tympanic membrane of the patient. In the treatment of keratosis obturans, the ear needle 24 with the cylindrical applicator 74 is preferred. With the desired ear needle 18, 22, 24 secured to the syringe distal end 42, the syringe is prepared for application of the pharmaceutical to the surface of the external ear canal.

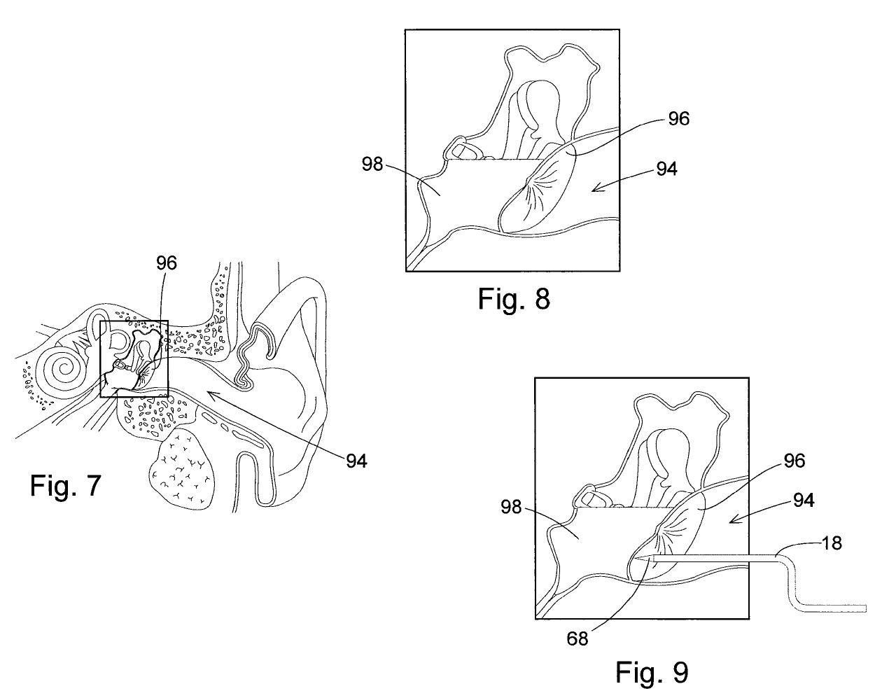

Application of the mitomycin-C to the tympanic membrane is illustrated in FIGS. 7-11. FIG. 7 shows a cross-section representation of the inner ear canal 94 and the tympanic membrane 96 or ear drum in the ear canal. FIG. 8 shows an enlarged view of the portion of the ear canals 94 shown in the rectangular box of FIG. 7. In FIG. 8, a fluid buildup 98 on one side of the ear drum 96 is a source of infection in the ear that is removed by the method of the invention.

FIG. 9 illustrates the application of the mitomycin-C to the tympanic membrane of the ear drum 96 by use of the absorbent pad 68 having the cone-shaped configuration. This pad 68 can be used to paint a location on the surface of the tympanic membrane 96 with the mitomycin-C absorbed in the pad 68.

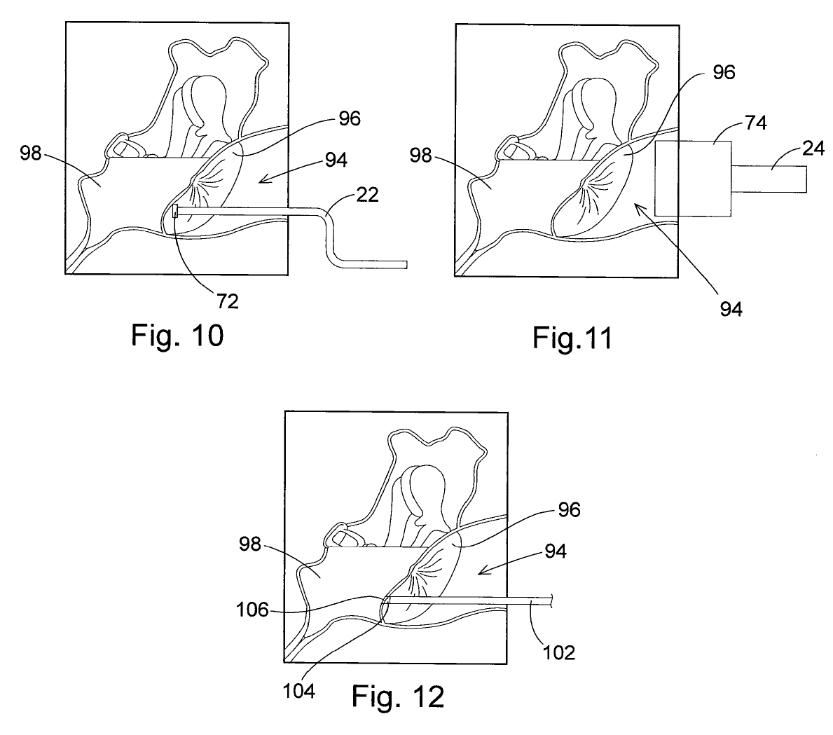

FIG. 10 illustrates the application of the mitomycin-C to a location on the tympanic membrane 96 using the planar, kidney-shaped configuration 72 of the absorbent pad. This configuration of the absorbent pad 72 can be used to stamp a location of application of the mitomycin-C on the tympanic membrane 96.

FIG. 11 illustrates the cylindrical-shaped absorbent pad 74 being used to apply the mitomycin-C to a location in the ear canal. The cylindrical-shaped pad 74 is used to apply the mitomycin-C to the circumference of the ear canal. In the treatment of keratosis obturans, the pad applicator is inserted into the external ear canal and conforms to the circumferential surface of the external ear canal. The mitomycin-C is supplied to the pad 74 by operation of the carrier syringe 44, and the mitomycin-C is applied to the surface of the external ear canal from which the prolific growth of endothelial cells has been removed.

Each of the absorbent pads 68, 72, 74 enables an application of a single dose of an otologic formulation of mitomycin-C to the ear tissue. Suspending a viscous agent or an adhesive agent in the mitomycin-C enables the mitomycin-C to remain in situ on the ear tissue and cause anti-metabolic activity on the tissue that is localized and defined by the specific color of the mitomycin-C.

A laser 102 that emits a laser beam 104 that is specifically tuned to the color of the mitomycin-C applied to the tympanic membrane 96 may then be used to ablate the tympanic membrane tissue at a location defined by the specific color of the mitomycin-C applied to the tissue. The application of the specifically tuned laser beam 104 to the tympanic membrane 96 is illustrated in FIG. 12. The laser beam 104 emitted from the laser 102 produces a photodynamic, selective, laser myringotomy 106. The effects of the mitomycin-C applied to the tympanic membrane 96 result in a sustained patency of the myringotomy 106 opening, produced by the laser beam 104.

Thus, the apparatus of the invention and its method of use discussed above enable a safe and simplified photodynamic laser myringotomy procedure that is not invasive and accommodates the non-compliant child patient population.

The apparatus of the invention and its method of use have been described above by reference to specific embodiments of the invention. It should be understood that modifications and variations could be made to the invention described without departing from the intended scope of the following claims.

1. An apparatus for application of a pharmaceutical to a surface of an external ear canal for treatment of keratosis obturans, the apparatus comprising:
the pharmaceutical being a single dose of an otologic formulation of mitomycin-C where the mitomycin-C when applied to the surface of the external ear canal causes anti-metabolic actively on the surface.

2. The apparatus of claim 1, further comprising:
the single dose of mitomycin-C being suspended in an agent that permeates living tissue with the mitomycin-C.

3. The apparatus of claim 1, further comprising:
the single dose of mitomycin-C being suspended in a viscous agent.

4. The apparatus of claim 1, further comprising:
the single dose of mitomycin-C being suspended in an adhesive agent.

5. The apparatus of claim 1, further comprising:
an ear needle that is adapted for receiving the single dose of mitomycin-C and delivering the mitomycin-C to the surface of the external ear canal to remain in situ on the surface and cause anti-metabolic actively on the surface that is localized.

6. The apparatus of claim 5, further comprising:
the ear needle having a tubular length with opposite proximal and distal ends, the needle proximal end being adapted for connecting and communicating the needle with a syringe, and the needle distal end having an absorbent flexible applicator fixed to the distal end.

7. The apparatus of claim 6, further comprising:
the applicator having a cylindrical shape that projects from the needle distal end.

8. The apparatus of claim 5, further comprising:
a tray;
a lid positionable over the tray;
the ear needle being positioned in the tray and covered by the lid;
a container of the single dose of mitomycin-C positioned in the tray and covered by the lid; and,
a syringe positioned in the tray and covered by the lid.

9. The apparatus of claim 8, further comprising:
the tray having at least first, second, and third separated compartments;
the ear needle being positioned in the first compartment of the tray;
the container being positioned in the second compartment of the tray; and,
the syringe being positioned in the third compartment of the tray.

10. An apparatus for application of a pharmaceutical to a surface of an external ear canal for treatment of keratosis obturans, the apparatus comprising:
the pharmaceutical being a single dose of an otologic formulation of mitomycin-C where the mitomycin-C, when applied to the surface of the external ear canal causes anti-metabolic actively on the surface of the external ear canal;
a delivery device having a length with opposite proximate and distal ends;
an absorbent applicator on the delivery device distal end, the absorbent applicator also being flexible for conforming to the surface of the external ear canal in response to the absorbent applicator on the delivery device distal end being inserted into the external ear canal.

11. The apparatus of claim 10, further comprising:
the delivery device being a tubular ear needle with an interior bore extending through the length of the delivery device, the interior bore being dimensioned to receive the single dose of the otologic formulation of mitomycin-C and to deliver the mitomycin-C to the absorbent applicator on the delivery device distal end.

12. The apparatus of claim 11, further comprising:
the delivery device proximal end being a proximal end of the ear needle, and the ear needle proximal end being connectable to a syringe.

13. The apparatus of claim 11, further comprising:
the delivery device distal end being a distal end of the ear needle, the absorbent applicator being on the ear needle distal end, and the absorbent applicator having a cylindrically shaped exterior surface that conforms to the surface of the external ear canal in response to the absorbent application being inserted into the external ear canal.

14. The apparatus of claim 11, further comprising:
a tray;
a lid positionable over the tray;
the ear needle being positioned in the tray and covered by the lid;
a container of the single dose of mitomycin-C positioned in the tray and covered by the lid; and,
a syringe positioned in the tray and covered by the lid.

15. The apparatus of claim 14, further comprising:
the tray having at least first, second, and third separated compartments;
the ear needle being positioned in the first compartment of the tray;
the container being positioned in the second compartment of the tray; and,
the syringe being positioned in the third compartment of the tray.

16. An apparatus for application of a pharmaceutical to a surface of an external ear canal for treatment of keratosis obturans, the apparatus comprising:
the pharmaceutical being a single dose of an otologic formulation of mitomycin-C where the mitomycin-C causes anti-metabolic activity on the surface of the external ear canal in response to application of the mitomycin-C to the surface of the external ear canal;
a delivery device that is operable to transmit the single dose of the otologic formulation of mitomycin-C to the surface of the external ear canal; and,
a sealed packaging containing the single dose of the otologic formulation of mitomycin-C and the delivery service.

17. The apparatus of claim 16, further comprising:
the packaging including a tray and a lid attached to the tray that is removable from the tray and is reattachable to the tray; and,
the delivery device being sealed in the tray with the lid attached to the tray.

18. The apparatus of claim 17, further comprising:
the delivery device being a tubular ear needle having a length with opposite proximal and distal ends, an interior bore extending through the ear needle length, and an applicator on the ear needle distal end that is operable to apply the single dose of the otologic formulation of mitomycin-C to the surface of the external ear canal.

19. The apparatus of claim 18, further comprising:
the applicator being absorbent and having a flexible exterior surface that is dimensioned to conform to the surface of the external ear canal in response to the applicator being positioned inside the external ear canal.

20. The apparatus of claim 19, further comprising:
a syringe adapted for containing the single dose of the otologic formulation of mitomycin-C; and,
the proximal end of the ear needle being connectable to the syringe, the syringe being sealed in the tray with the lid attached to the tray.

21. The apparatus of claim 20, further comprising:
the tray having at least first, second, and third separated compartments;
the single dose of the otologic formulation of mitomycin-C being positioned inside the first tray compartment;
the ear needle being positioned inside the second tray compartment; and,
the syringe being positioned inside the third tray compartment.

22. An apparatus for application of a pharmaceutical to a surface of an external ear canal for treatment of keratosis obturans, the apparatus comprising:
a container containing a single dose of an otologic formulation of mitomycin-C where the mitomycin-C when applied to the surface of the external ear canal causes an anti-metabolic actively on the surface of the external ear canal;
a syringe;
at least one ear needle having a tubular length with opposite proximal and distal ends, the proximal end of the ear needle being adapted for connection to and communication with the syringe;
at least one absorbent applicator adapted for communication with a distal end of an ear needle; and,
a packaging containing the container, the syringe, the ear needle, and the pad as a packaged kit.

23. The apparatus of claim 22, further comprising:
the packaging including a tray and a lid attached to the tray that is removable from the tray and is reattachable to the tray.

24. The apparatus of claim 23, further comprising:
the tray having at least first, second, and third separated compartments;
the container containing the single dose of the otologic formulation of mitomycin-C being positioned inside the first tray compartment;
the at least one ear needle being positioned inside the second tray compartment; and,
the syringe being positioned inside the third tray compartment.

25. A method for application of a pharmaceutical comprising:
providing a single dose of an otologic formulation of mitomycin-C; and,
applying the single dose of mitomycin-C to interior tissue of an ear canal.

26. The method of claim 25, further comprising:
applying the mitomycin-C to a surface of an external ear canal for treatment of keratosis obturans.

27. The method of claim 25, further comprising:
suspending the mitomycin-C in a viscous agent.

28. The method of claim 25, further comprising:
suspending the mitomycin-C in an adhesive agent that can permeate living tissue.

29. The method of claim 25, further comprising:
causing anti-metabolic actively on a surface of an external ear canal by application of the mitomycin-C at a location on the surface of the external ear canal.
